(19) United States
(12) Patent Application Publication
(10) Pub. No.: US 2009/0043282 A1
(43) Pub. Date: Feb. 12, 2009

(54) Drug Delivery Devices and Related Components, Systems and Methods

(75) Inventors: Martin Hughes, Wavendon (GB); Darren Seymour, Hartford (GB); Colin Turner, Girton (GB); Paul Harmer, Comberton (GB); Yannick Hourmand, Great Eversden (GB)

Correspondence Address: FISH & RICHARDSON P.C., P.O BOX 1022, MINNEAPOLIS, MN, 55440-1022, US

(73) Assignee: WYETH, Madison, NJ (US)

(21) Appl. No.: 11/912,003

(22) PCT Filed: Apr. 26, 2006
(86) PCT No.: PCT/US06/15709

§ 371 Date: Oct. 19, 2007

Related U.S. Application Data
(60) Provisional application No. 60/675,995, filed on Apr. 29, 2005.

(51) Int. Cl. A61M 5/31; A61B 17/58

(52) U.S. Cl. 604/518; 604/82; 604/83; 606/93

(57) ABSTRACT

This description relates to drug delivery devices (100), as well as related components, systems and methods.

### TECHNICAL FIELD

This description relates to drug delivery devices, as well as related components, systems and methods.

### BACKGROUND

Calcium phosphate-based cement is commonly used in many orthopedic and anaplastic surgical procedures. Various devices have been developed to prepare and/or deliver bone cement in such procedures.

### SUMMARY

This description relates to drug delivery devices, as well as related components, systems and methods.

In various embodiments, the drug delivery devices, systems and methods can offer a reliable, repeatable, and/or consistent delivery of a predetermined volume of a liquid containing a therapeutic agent, such as an osteogenic agent. Prior to dissolution in the liquid, the therapeutic agent can be provided (stored) in the delivery device, for example, in solid (e.g., powder) form. Examples of therapeutic agents include proteins, such as a member of the transforming growth factor beta (TGF-β) family, at least one protein from the bone morphogenetic protein (BMP) family of proteins, or at least one protein from the growth/differentiation factor (GDF) family of proteins. In some embodiments, the therapeutic agent includes combinations of proteins, for example, combinations of any of the foregoing proteins.

The compound can be secured in the device until reconstituted and administered to a patient to help control (e.g., prohibit) unintended usage. The components for reconstituting, mixing and agitating and delivery the admixture can be aseptically contained within a unitary system, thereby minimizing or eliminating contamination. The delivery device can also provide force enhancement to mix and prepare for injection reconstituted compounds which exhibit substantially viscous properties.

In one aspect, a drug delivery system features a drug delivery device including a main body including a proximal end, a distal end, and a mixing chamber positioned between both ends, a rotary driver disposed at the proximal end of the main body, a main piston operably linked to the rotary driver, an agitator disposed with the mixing chamber and affixed to an agitator shaft, the agitator shaft operably linked to the main piston such that rotating the rotary drive imparts axial movement to agitator, and a piston end operably linked to the main piston.

In another aspect, a drug delivery system features a delivery device, a reconstitution manifold and an air pump. The reconstitution manifold includes a first vial to contain a first substance, a second vial, in fluid communication with the first vial, to contain a second substance. The air pump is in fluid communication with the first and second vials and configured to operate in at least a first and second mode. While operating in the first mode, at least part of the first substance is combined with the second substance to form a resulting admixture. While operating in the second mode, the admixture is transferred from either the first or second vials to the delivery device.

In another aspect, a method for preparing calcium phosphate-based cement includes reconstituting a BMP powder to form a BMP admixture in manifold, delivering the BMP admixture from the manifold to a delivery device releasably attached to the manifold, mixing the admixture with a CPM (calcium phosphate matrix) contained within the delivery device to form a third substance, and displacing a piston slidably disposed within the delivery device to eject the third substance from the delivery device.

Features and advantages will be apparent from the description, drawings and claims.

### DESCRIPTION OF DRAWINGS

FIGS. 1A and 1B are perspective and side views of an embodiment of a drug delivery device, respectively.

FIGS. 2A and 2B are perspective and side views of an embodiment of a drug delivery device, respectively.

FIG. 3 is a partial sectional perspective view of the device of FIGS. 1A and 1B.

FIG. 4 is an exploded and partial sectional view of the device of FIGS. 1A and 1B.

FIGS. 5 and 6 are detailed perspective view of components disposed at the distal end of the device of FIGS. 1A and 1B.

FIG. 7 is a detailed perspective view of components disposed at the proximal end of the device of FIGS. 2A and 2B.

FIG. 8A is a cross-sectional view of the device of FIGS. 1A and 1B.

FIG. 8B is a detailed view of the area 8B of FIG. 8A.

FIG. 9 is a cross-section of view of the device of FIGS. 2A and 2B.

FIG. 10 is a graphical depiction of the axial and rotation movement of components of the devices of FIGS. 1A through 2B.

FIG. 11 is a detailed view of an agitator of the devices of FIGS. 1A through 2B.

FIG. 12 is a schematic view of a drug delivery system including the drug delivery device of FIGS. 1A and 1B and a reconstitution manifold.

FIG. 13 is a perspective view of a drug delivery system.

FIGS. 14A and 14B are partial sectional perspective views of the drug delivery system of FIG. 13.

FIG. 15 is a perspective view of the internal components of the reconstitution manifold of FIG. 13.

FIG. 16 is a cross-sectional view of internal components of the reconstitution manifold of FIG. 13.

Like reference symbols in the various drawings indicate like elements.

### DETAILED DESCRIPTION

In certain embodiments, a drug delivery system reconstitutes a first substance, mixes and agitates the reconstituted first substance with a second substance to form a third substance and delivers, by injection, the third substance to a patient.

In some embodiments, the first substance is a therapeutic agent, such as an osteogenic agent. A therapeutic agent can be provided, for example, in solid (e.g., powder) form. Examples of therapeutic agents include proteins, such as members of the TGF-β family (e.g., one or more members of the BMP family of proteins, one or more members of the GDF family of proteins). Examples of osteogenic agents are disclosed, for example in U.S. Pat. Nos. 6,719,968, 6,027,919, 5,658,882, 5,618,924 and 5,013,649, which are hereby incorporated by reference. In certain embodiments, the osteogenic agent is BMP-2, BMP-12 or MP52. In some embodiments, multiple therapeutic (e.g., osteogenic) agents can be used. In certain embodiments, the second substance is a CPM powder.

In general, the first substance is reconstituted and transferred to a delivery device containing the second substance wherein the reconstituted BMP powder and CPM are mixed to form a third substance. The third substance is then ejected from the delivery device and administered to a patient by injection, for example.

In some embodiments, the drug delivered system is configured for preparing a homogeneous third substance, which includes the first and second substances, and has physical properties such as viscosity, density and specific gravity well suited for delivery to a patient by injection, for example. In some embodiments, the device is configured for use with BMP-2.

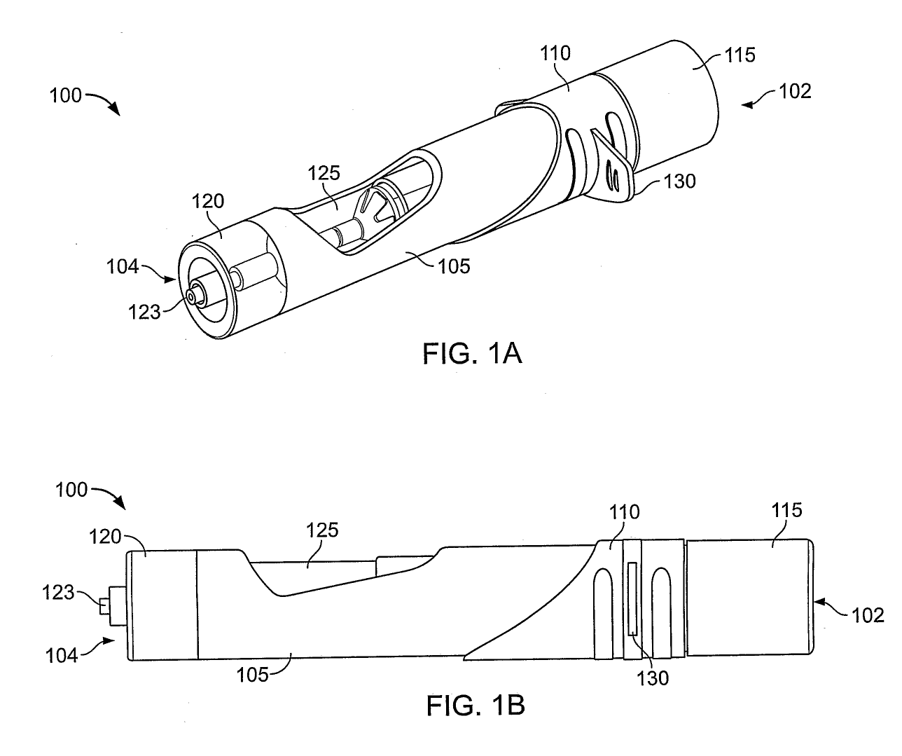

FIGS. 1A and 1B show a delivery device 100 having a proximal end 102 and a distal end 104 and including a main body front 105 joined to an axially extending main body rear 110. The delivery device can be dimensioned to administer a suitable quantity, such as 5-ml or 1-ml of a material, such as a bone cement, to a patient. A rotary drive 115 is threadably attached at the proximal end 102 of the main body rear 110 and an outer front 120 is threadably attached to the main body front 105. The outer front 120 includes a luer connection 123 configured to receive a needle or reconstitution manifold (described below). In certain embodiments, the main body front 105 includes a window 125 to display the position of the interior components of the device 100 and the contents therein. In some embodiments, the main body rear 115 includes laterally extending fins 130.

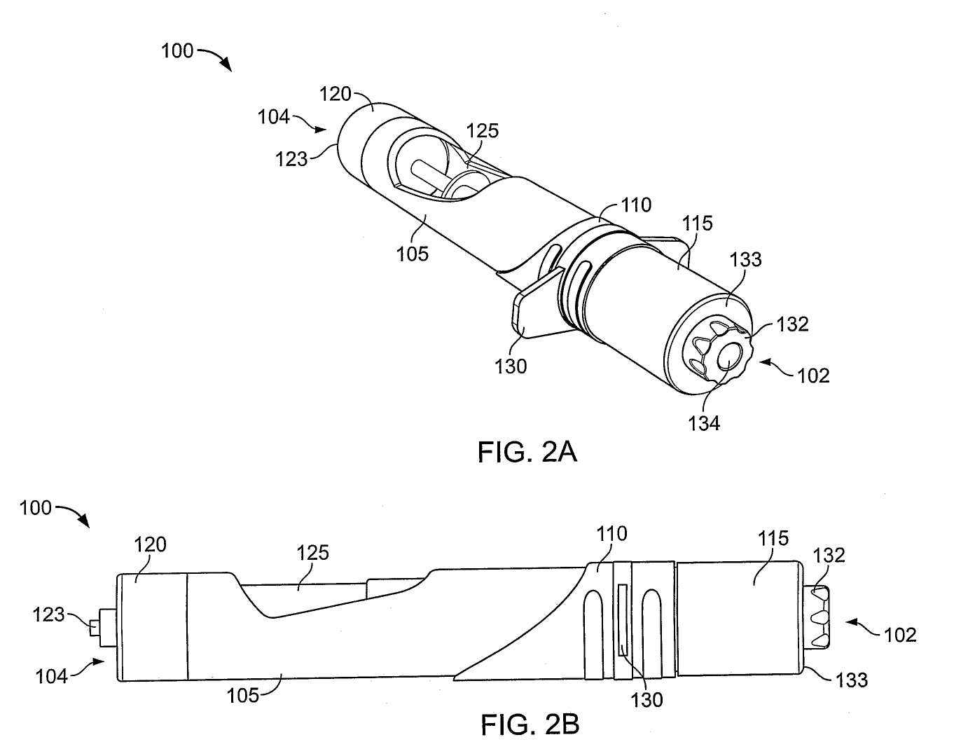

In some embodiments, as shown in FIGS. 2A and 2B, the delivery device 100 can include a piston end 132 extending along a concentric bore in the rotary drive 115 and protrudes beyond a proximal end 133 of the drive 115.

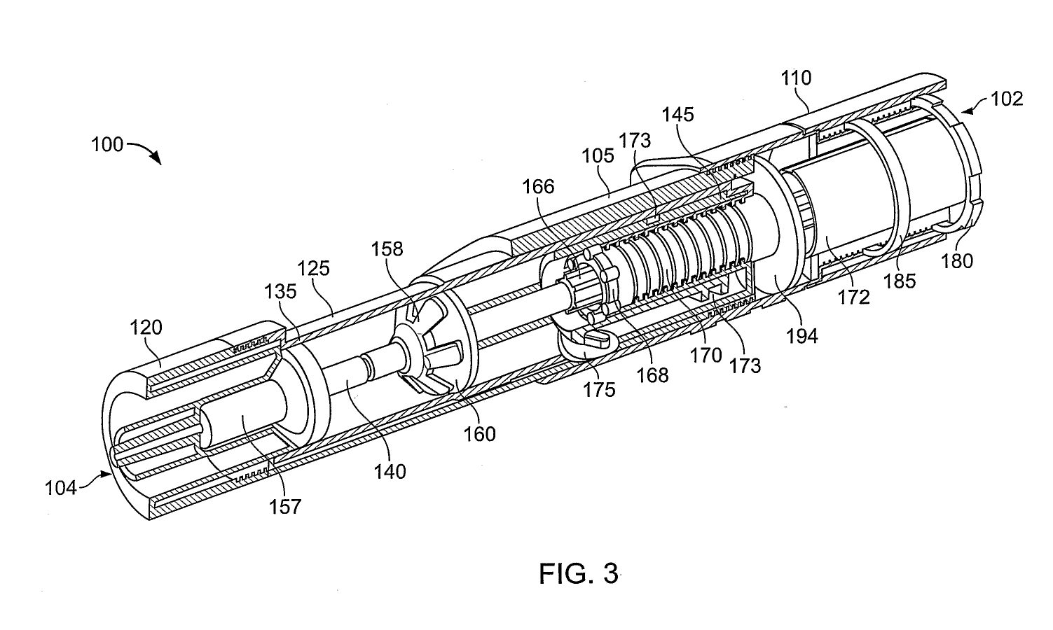

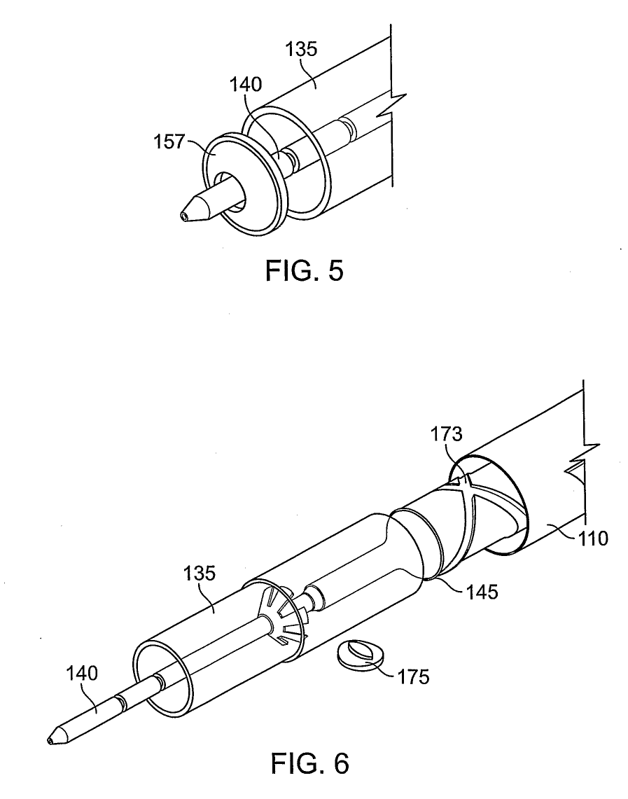

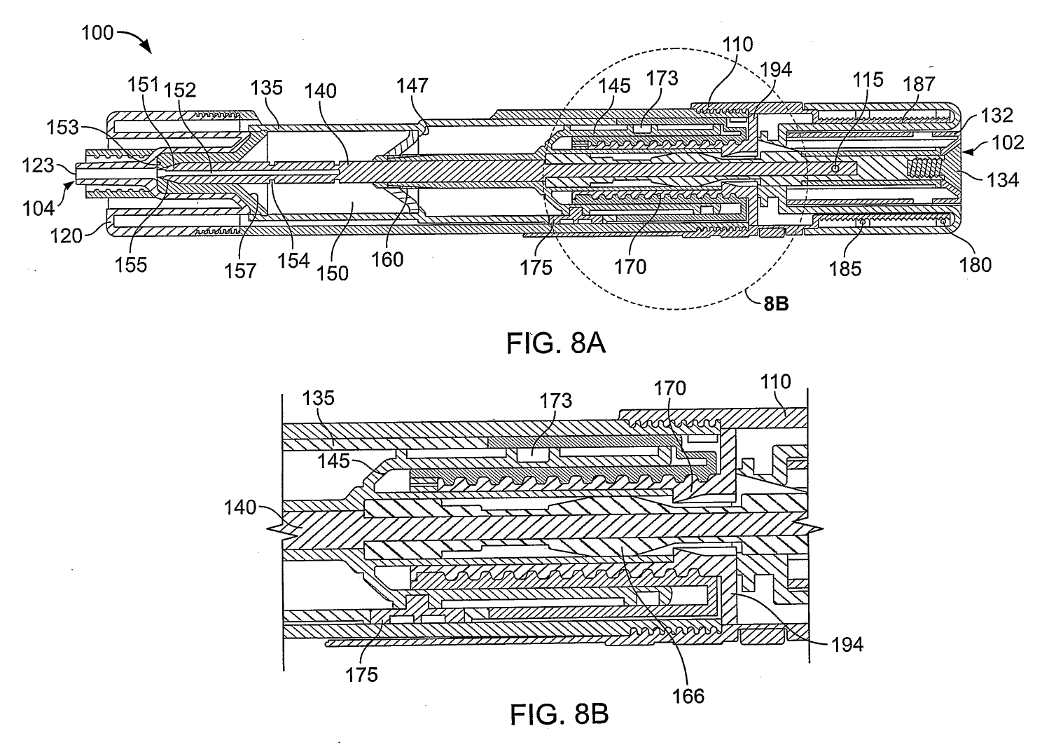

FIGS. 3 through 9 show the internal components of the delivery device 100 including a syringe barrel 135 disposed within the main body front 105. A piston shaft 140 extends axially along the length of the device between the proximal and distal ends 102, 104. An agitator shaft 145 concentrically receives a portion of the piston shaft 140. The syringe barrel 135 includes a frustoconical wall 147 (FIGS. 8A and 8B) to define a mixing chamber 150 through which the piston shaft 140 extends. The syringe barrel 135 can include a circumferentially extending vent (not shown) to allow evolved gases within the barrel to escape while the mixing chamber 150 is filled and/or during the mixing and/or ejection of the contents contained therein (described below). The vent can be formed from a sintered material wrapped with a high density polyethylene, such as polytetrafluoroethylene (PTFE), for example.

The piston shaft 140 includes a piston head 151 and an axial central bore 152 extending along the piston shaft 140 from a port 153 located at a distal end of the piston head 151, toward the mixing chamber 150, when the piston shaft 140 is in a filly distal position. The central bore 152 can includes transverse ports 154, such that when the piston head 151 is in a filly distal position, the luer connection 123 is in fluid communication with the mixing chamber 150.

The outer front 120 can include a substantially cylindrical ejection chamber 155 which receives a front seal 157. The ejection chamber 155 and front seal 157 are sized to receive the piston head 151 of the piston shaft 140. In some embodiments, the mixing chamber 150 is preloaded with the second component, such as, for example, a CPM powder, for mixing with the first component, such as, for example, a reconstituted BMP liquid that is introduced through the luer connection 123, along the central bore 152, out of the transverse ports 154 and into the syringe barrel 135.

In some embodiments, the transverse ports 154 include a circular seal (not shown), such as an o-ring, for example, which permits the passage of fluid from the central bore 152 to the mixing chamber 150 but substantially impedes the reverse flow of fluid from the mixing chamber 150 to the central bore 152. In certain embodiments, the circular seals allow a reconstituted BMP-2 liquid to flow into the mixing chamber 150 via the transverse ports 154 and prevents the BMP-2 liquid from flowing back into the central bore 152 during the mixing and ejection of the BMP-2 liquid with the CPM. The port 153 at the distal end of the piston head 151 can also include a plug (not shown) of sintered material which is configured to allow the passage of a liquid, such as, for example, the flow of reconstituted BMP-2 through the luer connection 123 and into the central bore 152, but substantially impedes the flow of a paste, such as, for example, a mixture of reconstituted BMP-2 and CPM.

An agitator 158 and an agitator seal 160 which seated proximally to the agitator 158 and can be frustoconical, for example, are contained within the mixing chamber 150 and are attached to the piston shaft 140. A piston end 132 (FIGS. 8A and 8B) is attached to a proximal end of the piston shaft 140 with a fastener 134.

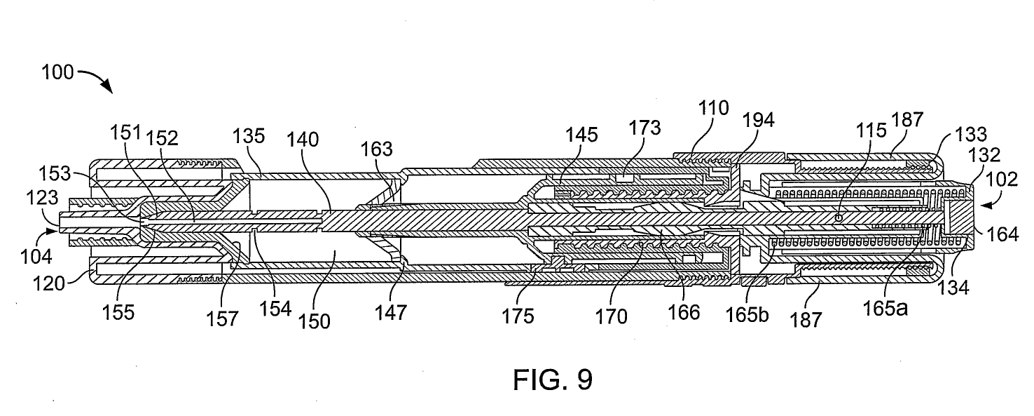

In certain embodiments, shown in FIG. 9, an agitator and an agitator seal integrally form an agitator assembly 163. The agitator assembly 163 engages the frustoconical wall 147 of the syringe barrel 135 to form a seal therewith. The proximal end 164 of the piston shaft 140 extends and connects with the fastener 134. A piston end spring 165a and main piston spring 165b bias the piston shaft 140 distally to improve contact between the piston head 151 with the front seal 157 during mixing.

The piston end 132 is configured for axial (translational) movement and the rotary drive 115 is configured for rotational movement. A main piston 166, an inner ratchet 168, a drive inner 170 and a drive outer 172 are concentrically arranged along the piston shaft 140 between the agitator shaft 145 and the main body rear 110 to translate the rotary movement of the rotary drive 115 to the agitator 158 and the axial movement of the piston end 132 to the main piston 166 as described below.

A cam track 173 extends around the agitator shaft 145 and receives a cam follower 175 mounted to an inner surface of the syringe barrel 135. The rotary drive 115 is keyed directly to the main piston 166 proximate the piston end 132 (FIGS. 8A and 8B). Accordingly, rotation of rotary drive 115 rotates the main piston 166. The main piston 166 is keyed to the agitator shaft 145, such that as the main piston 166 rotates, engagement of the cam follower 175 with the cam track 173 imparts axial movement of the agitator shaft 145 and the attached agitator 158 within the mixing chamber 150. The cam track 173 can describe a helical path, for example, about the agitator shaft 145. A retainer ring 177, a drive runner 180, and a drive runner stop ring 185 are concentrically arranged along the drive outer 172 between the main body rear 110 and the rotary drive 115.

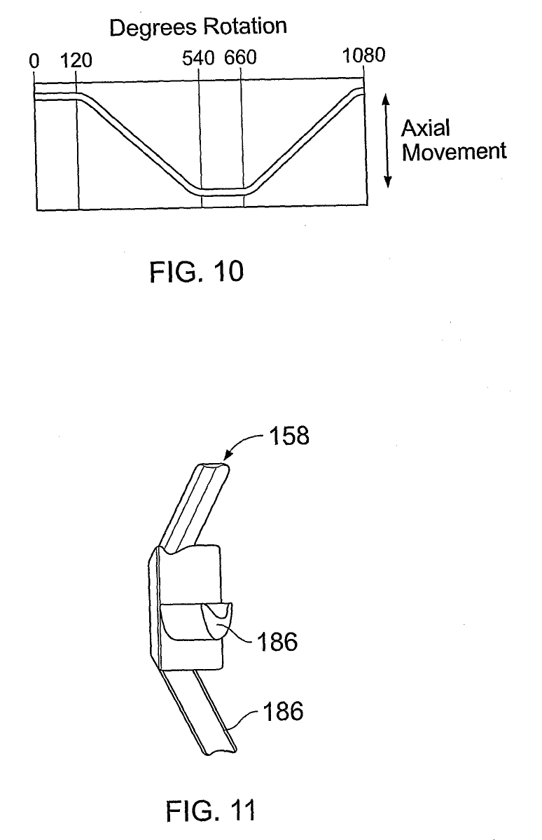

In operation, in certain embodiments, the agitator shaft 145 and attached agitator 158, follow a reciprocating motion within the mixing chamber 150 to mix the paste. Referring to FIG. 10, one prescribed motion includes 120-degrees rotation of the rotary drive 150; a 240-degrees helical motion with 30-mm axial movement towards the distal end 104; a 120-degrees rotation; and 240-degrees helical motion with 30 mm axial movement towards the proximal end 102. The agitator 158 thus returns to its starting point every two revolutions of the rotary drive 115.

In certain embodiments, as shown in FIG. 11 and, the agitator 158 includes a plurality of blades 186 extended substantially radially from the agitator shaft 145. The blades 186 can be made from a variety of material including, for example, polycarbonate, Santoprene® (Monsanto Corporation, Delaware), polyester elastomer, polypropylene, or polyethylene. The blades 186 can be formed from a flexible material and configured to mix and agitate the contents of the mixing chamber 150 to form a homogeneous paste. The blades 186 can be configured to deform when the agitator shaft 145 is moved rotationally and remain radially extended when the agitator shaft 145 is moved axially.

As the agitator 158 or agitator assembly 163 (FIG. 9) operates, the drive inner 170 and drive outer 172 components will also rotate within the main body front 105. The rotary drive 115 is keyed to a drive runner 180, which engages threads 187 on the main body rear 110. As the rotary drive 115 is turned, the drive runner 180 moves along the main body rear until it contacts the drive runner stop ring 185. The retainer ring 177 includes two tabs 188 configured to engage slots 189 disposed in the main body rear 110 proximal to the fins 130, to prevent the rotary drive 115 from moving axially relative to the main rear body 110. The drive runner stop ring 185 is pinned to the main body rear 110 and secured in place.

The drive runner stop ring 185 includes circumferentially located bosses 190 sized and configured to engage circumferentially located recesses 193 on the drive runner 180. As the rotary drive 115 and agitator 158 are turned to mix the contents of the mixing chamber 115, the drive runner 180 advances axially along threads 187 a predetermined distance until the drive runner is proximate the drive runner stop ring 185. The bosses 190 along the drive runner stop ring 185 engage the recesses 193 along the drive runner 180 locking the drive runner 180 against further rotation. In some embodiments, after about 16-turns of the rotary drive 115 and a satisfactory mix of the contents of the mixing chamber 150 is achieved, the drive runner 180 is locked to the drive runner stop ring 185.

After the contents of the mixing chamber 150 are sufficiently mixed, the rotary drive 115 is locked against rotation by the driver runner stop ring 185. The agitator shaft 145 is therefore prevented from rotating in the main body front 105. The piston end 132 is released by rotating it clockwise relative to the rotary drive 115 (when viewed from the proximal end 102), which unlatches a bayonet arrangement (not shown) between these two parts. The piston end 132 and the main piston 166 are then urged back (towards the proximal end) by about 30 mm under the action of a spring (not shown).

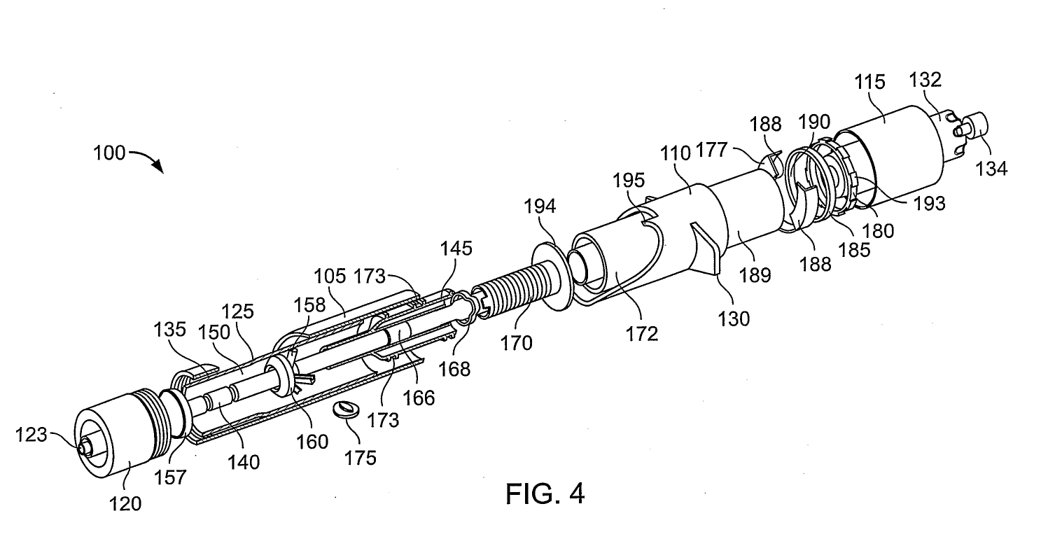

The drive inner 170 is configured to rotate and be fixed axially by an radially extending flange 194 disposed between the main body front 105 and the rotary drive 115. The drive inner 170 connects to the main piston 166 by a two start 120-mm pitch thread, and therefore rotates by 90-degrees counterclockwise as the main piston 166 moves back. The drive outer 172 carries a set of ratchet arms 195 (FIG. 4) that permit only clockwise rotation within the main body front 105 and is threaded to the drive inner 170 via a single start 3-mm pitch thread. Therefore, as the drive inner 170 rotates counterclockwise, the drive outer 172 is forced to rotate 90-degrees relative to the drive inner 170, and is pushed 0.75 mm towards the distal end of the device.

After the piston end 132 is depressed a full stroke by the user (in the distal direction), the piston end 132 then moves back toward its original position (in the proximal direction) to complete a return stroke under the action of the a spring (not shown) located in an annular region between piston end 132 and the rotary drive 115. As the piston end 132 is depressed by the operator, the drive outer 172 bears directly on the syringe barrel 135 such that the syringe barrel 135 is forced 0.75-mm towards the distal end 104 of the device. The operator can use the laterally extending fins 130 (FIGS. 1A through 2B) for improved leverage in depressing the piston end 132.

In some embodiments, a full return stroke of the piston end 132 in the proximal direction, 30-mm, for example, translates into an axial movement of the syringe barrel 135 of only 0.75-mm, while increasing the transmitted axial force by a factor of 40 (30 /0.75-mm). Such force enhancement decreases the static and dynamic force requirements for mixing and displacing the contents of the syringe barrel. This is particularly advantageous when the syringe barrel 135 contains a substantially viscous fluid. The force enhancement and corresponding axial advancement of the syringe barrel 135 can be modified to suit various operator force requirements and fluid viscosities.

The piston end 132 is pushed against the load of the spring (not shown) and the drive inner 170 rotates 90 degrees clockwise. The drive outer 172 is connected to the drive inner 170 via a set of ratchet arms that permit the drive outer 172 to only rotate clockwise relative to the drive inner 170. Therefore, as the drive inner 170 rotates clockwise it carries the drive outer 172 with it, rotating in the main body front 105.

The syringe barrel 135 remains stationary relative to the main body front 105 during the forward stroke of the main piston 166, and the front of the main piston 166 moves into a reduced diameter section of the outer front 120, which serves as a small diameter paste dispensing syringe.

In certain embodiments, the volume occupied by mixed paste contained within the mixing chamber 150 is less than that occupied by the CPM powder, so there is a void within the mixing chamber at the end of the mixing process. The first 10 to 15 strokes of the piston end 132 and the main piston 166 serve to take up the void space.

After the void is filled by the movement of the syringe barrel 135 relative to the outer front 120, the reducing volume of the mixing chamber 150 causes the contents of the mixing chamber 150, such as a paste for example, to flow into the reduced diameter bore in the outer front seal 157, as the main piston 166 is withdrawn. On the subsequent advance of the main piston 166, this paste is forced out of the luer connection 123 on the front of the outer front 120, and into an injection needle attached to the luer connection 123. In certain embodiments, the volume of paste ejected from the device is from about 0.1 ml to about 0.3 ml per stroke of the main piston 166.

The outer front 120 is connected to the main body front 105 and slides within the syringe barrel 135, so the effect of the movement of the syringe barrel 135 is to reduce the axial length of the mixing chamber 150. The movement of the outer front 120 at the distal end of the mixing chamber 150, addresses a phenomenon referred to as filter pressing, whereby the liquid in a multiphase composition, such as a calcium phosphate cement, for example, separates from the solid at the point where the load is applied, thereby leaving the solid portion behind, during ejection from a syringe, for example. In certain embodiments, the application of the load by the movement of the syringe barrel 135 relative to the outer front 120 proximate the outlet of the mixing chamber 150, i.e., the luer connection 123, helps the portion of the paste proximate the luer connection 123 to remain dry and the general body of the paste retain a sufficiently high water content for subsequent ejection and delivery through the luer connection 123.

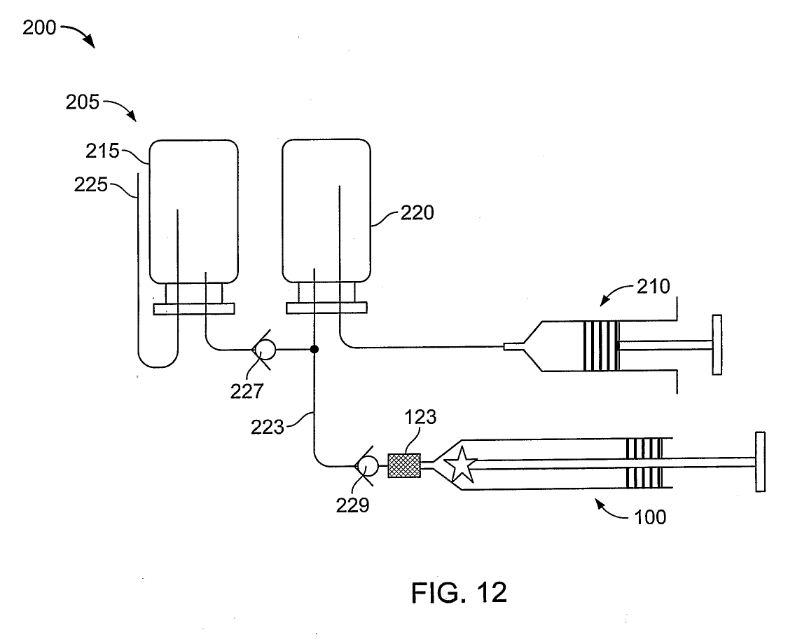

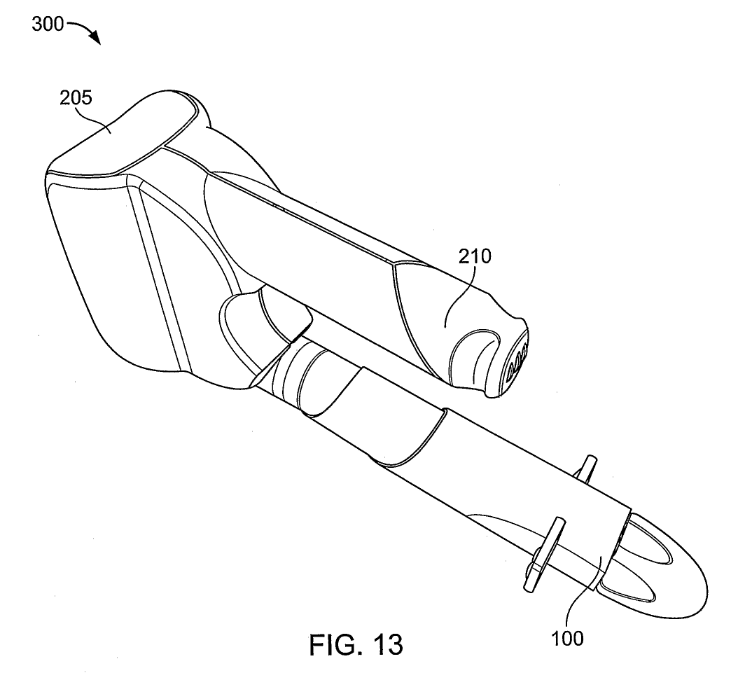

Referring generally to FIGS. 12-16, the delivery device 100 can be used as a component in a drug delivery system 200 which also includes a reconstitution manifold 205 and a syringe 210 which can also include an air pump or other pressure source. In certain embodiments, the manifold 205 includes two vials, a water for injection (WFI) vial 215 and a concentrate vial 220. The contents of the vials 215, 220 form an admixture to be delivered through an exit port 223 which is releasably attached to the luer connection 123 of the outer front 120 of the device 100. The WFI vial 215 includes a vent 225 extending generally upwards and terminating in a catch-pot (not shown) which is open to ambient and has a volumetric capacity substantially equal to the volume of the vent 225.

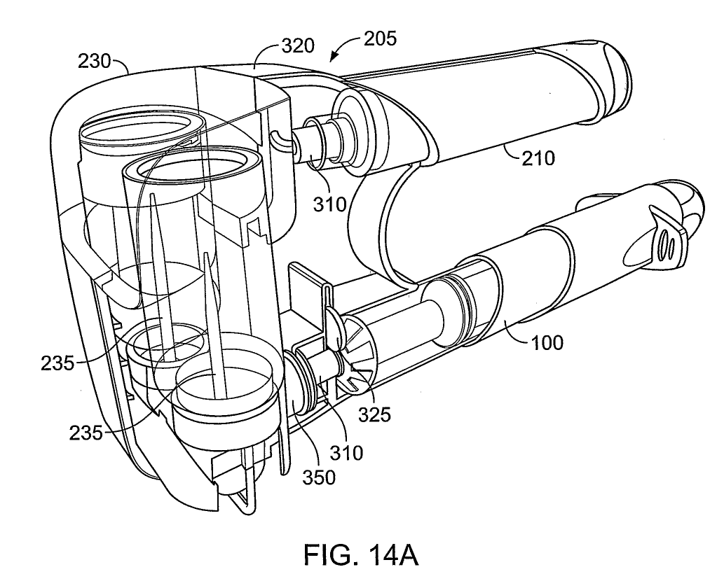

FIG. 13 depicts a unitary system 300 including components of the drug delivery system 200. The drug delivery device 100 and/or the syringe 210 can be releasably attached to the reconstitution manifold 205 during varying stages of use of the drug delivery system 200.

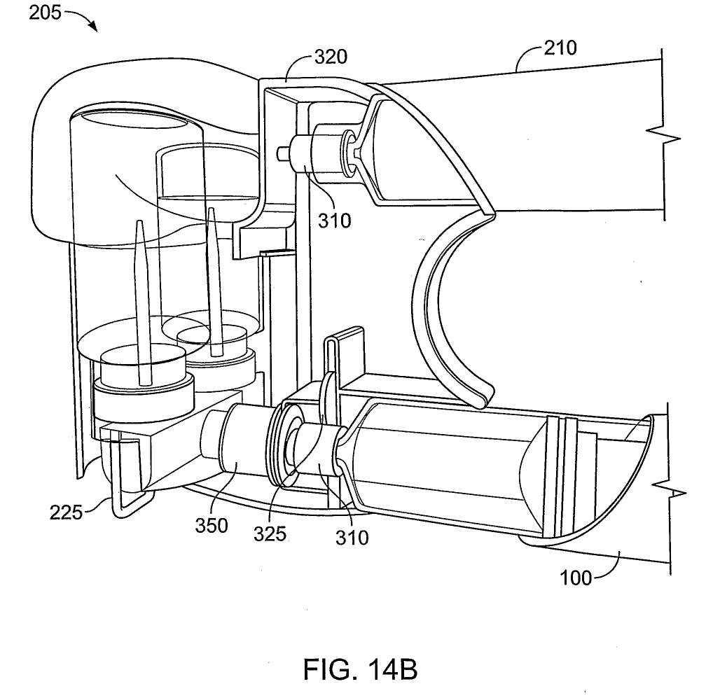

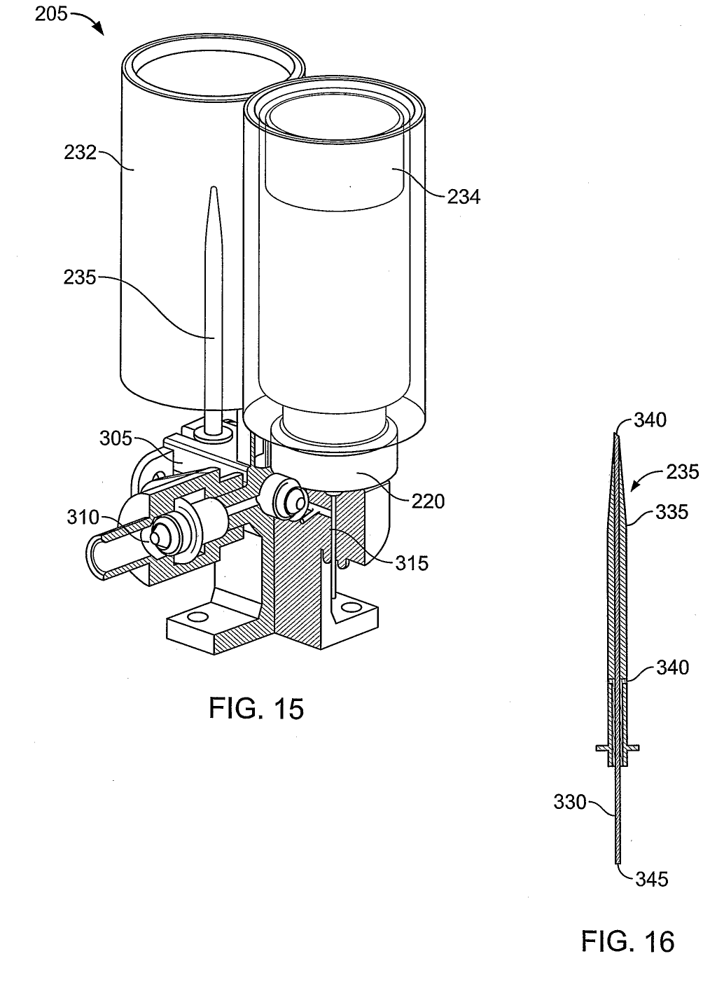

With specific reference to FIGS. 14A through 15, the manifold 205 can include a cover 230 slidably disposed within the manifold 205 and configured to position vials 215 and 220 above concentric needles 235 in a raised position. In one embodiment, the vials 215, 220 are locked inside of the cover 230 to limit access and the unintended use of the vial contents, before transfer to the delivery device 200 as described below. The cover 230 can be substantially transparent to reveal the enclosed vials 215 and 220. When the cover 230 is pushed toward a lowered position, the vials 215 and 220 are pierced by the concentric needles 235 for evacuation of the vial contents during operation of the manifold 205.

The cover 230 can include vial guides 232, 234 (FIG. 15) to center the vials 215, 220 on the concentric needles 235. The manifold 205 can include a manifold assembly 305 to support the vials 215, 220 as the cover 230 is moved to the lowered position. The delivery device 100 is attached to the manifold 205 at a device connector 310 (FIGS. 14A and 14B). The syringe 210 is attached to the manifold 205 at an air pump connector 315. In certain embodiments, all components of the manifold 205, including the vials 215, 220 are self-contained by an external housing 320 which can be configured to be tamper-evident or tamper-proof. A connection shutter 325 is slideably disposed proximate the device connector 310 and configured to be normally held open against the bias of a spring (not shown) by the luer connection 123 when the delivery device 100 engages the manifold 205. When the delivery device 100 is removed from the manifold 205, the connection shutter 325 springs closed and passes into a groove (not shown) configured such that the shutter 325 can not subsequently be reopened. This configuration minimizes the possibility of extraction of the contents of the vials 215, 220 from the manifold 205 by premature removal of the delivery device 100.

As shown in FIG. 16, the concentric needles 235 can include a core needle 330 substantially surrounded by an outer sheath 335 to define annular space between the core needle 330 and the sheath 335 for passage of the contents of the vials to the manifold 205. The core needle 330 is open at a needle tip 340 and connected to an air conduit 345 to provide for passage of air and between the vial 215 and the air pump 210 and between the vial 220 and the vent 225. Transverse ports 340 extend from the sheath 335 to provide fluid communication between the vials 210, 215 and the manifold 205.

In use, an operator pushes the cover 230 of the manifold 205 downward thereby penetrating the vials 215, 220 with concentric needles 235. The syringe 210 is first pulled out to draw the WFI from the vial 215 through a first one-way valve 227 and into the concentrate vial 220, facilitated by the vent 225. The manifold 205 is then gently agitated to reconstitute the contents of the concentrate vial 220. The syringe 210 is then pushed in to force the reconstituted mixture from the concentrate vial 220 through a second one-way valve 229 and into the delivery device 100. The delivery device 100 can be removed from the manifold 205 by rotating the delivery device 100, for example, and detaching the luer connection 123 from the device connector 310. The connection shutter 325 closes to limit access to the remaining contents of the concentrate vial 220. Both of the return valves 227, 229 and the luer connection 123 can be contained with a valve manifold 350.

The operator then rotates the rotary drive 115 clockwise (as viewed from the proximal end 102) about 15 or 16-full rotations, for example, to form a paste in the mixing chamber 150 of the delivery device 100 and lock the rotary drive 115. Accordingly, the paste can be consistently, uniformly and aseptically mixed within the mixing chamber 150 before delivery to the ejection chamber 155 and passage through the luer connection 123. A delivery needle (not shown), such as a Tuohy needle with an obdurator, for example, is connected to the luer connection 123 of the delivery device 100. The delivery needle can be positioned within a patient before or after connection with the delivery device 100, using a fluoroscope, for example, and directed at a treatment site, such as a wrist or hip, for delivery of the contents of the mixing chamber 150 of the delivery device 100.

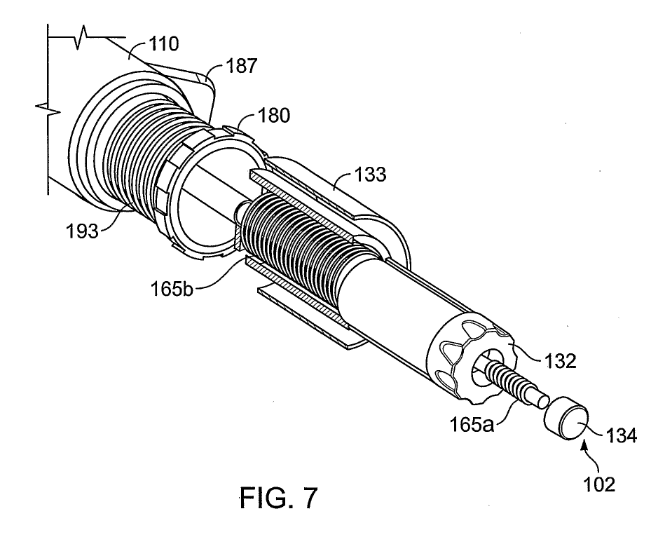

The rotary driver 115 or the piston end 132 protruding from the rotary drive 115 (FIGS. 8A, 8B, and 9) is rotated clockwise (as viewed from the proximal end 102) 15 to 20-degrees, for example, to release the bayonet and force the piston end 132 in a proximal direction under the bias of the springs 165a and 165b (FIGS. 7, and 9). The piston end 132 rotates independently of the rotary drive 115.

The operator then depresses the piston end 132 in a distal direction 10 to 15 full strokes, for example, before the paste is available for ejection from the ejection chamber 155, through the luer connection 123 and the connected delivery needle, and injection to the treatment site. In some embodiments, the delivery device 100 is configured for one-time usage.

While certain embodiments have been described, others are possible.

As an example, while certain dimensions have been disclosed, in general any desired dimensions can be used.

As another example, while formulation and delivery of bone cement have been described, other mixtures can also be formed and/or delivered.

As a further example, while certain applications of systems and devices have been described, in general, the devices and systems can be used in any desired application. As an example, the devices and systems can be used in tissue (e.g., bone, cartilage, tendon, meniscus, ligament) treatment and/or repair. In some embodiments, the devices and systems can be used in bone-to-bone repair. In certain embodiments, the devices and systems can be used in cartilage regeneration. In some embodiments, the devices and systems can be used in bone fracture repair. Additionally or alternatively, the devices and systems can be used in implant treatment and/or repair. As an example, the devices and systems can be used to grout one or more implants.

Other embodiments are in the claims.

What is claimed is:

1. A drug delivery device, comprising:
an agitator assembly disposed within a mixing chamber defined by a tubular member of the drug delivery device, the agitator assembly being configured to axially reciprocate within the mixing chamber.

2. The drug delivery device of claim 1, wherein the delivery device is configured to prevent further axial reciprocation of the agitator assembly after a predetermined number of cycles of axial reciprocation.

3. The drug delivery device of claim 1, wherein the agitator assembly is operably connected to a rotary drive such that rotation of the rotary drive causes the axial reciprocation of the agitator assembly.

4. The drug delivery device of claim 3, wherein rotation of the rotary drive in a single direction causes the axial reciprocation of the agitator assembly.

5. The drug delivery device of claim 3, wherein the rotary drive is configured to rotatably lock after a predetermined number of rotations.

6. The drug delivery device of claim 5, wherein the rotary drive comprises a projection extending therefrom, the projection being arranged to mate with a recess defined in a substantially rotatably fixed component of the drug delivery device after the predetermined number of rotations.

7. The drug delivery device of claim 6, wherein the projection extends from a drive runner connected to the rotary drive, and the substantially rotatably fixed component comprises a ring extending about the drug delivery device.

8. The drug delivery device of claim 1, wherein the agitator assembly is further configured to rotate within the mixing chamber.

9. The drug delivery device of claim 1, wherein the agitator assembly is configured to reciprocate in a substantially helical motion within the mixing chamber.

10. The drug delivery device of claim 1, wherein the agitator assembly comprises an agitator shaft and an agitator secured to the agitator shaft.

11. The drug delivery device of claim 10, wherein the agitator comprises a plurality of projections extending radially from the agitator shaft.

12. The drug delivery device of claim 1, wherein the tubular member of the drug delivery device comprises a radial projection, and the agitator shaft comprises a channel helically extending around an exterior surface thereof and configured to receive the projection.

13. The drug delivery device of claim 12, wherein the projection extends radially inward from an inner surface of the tubular member.

14. The drug delivery device of claim 1, further comprising a therapeutic agent disposed within the mixing chamber.

15. The drug delivery device of claim 14, wherein the therapeutic agent comprises a powdered substance.

16. The drug delivery device of claim 14, wherein the therapeutic agent comprises an osteogenic agent.

17. The drug delivery device of claim 1, further comprising a piston assembly operably connected to the tubular member and configured to axially displace the tubular member relative to an axially fixed distal member of the drug delivery device.

18. The drug delivery device of claim 17, wherein the piston assembly is configured such that an axial force applied to the piston assembly causes an axial force to act on the tubular member, the axial force acting on the tubular member being greater than the axial force applied to the piston assembly.

19. The drug delivery device of claim 18, wherein the axial force acting on the tubular member is greater than the axial force applied to the piston end by a factor of about 40.

20. The drug delivery device of claim 17, wherein the piston assembly is configured such that an axial displacement of the piston assembly causes axial displacement of the tubular member, the axial displacement of the piston assembly being greater than the axial displacement of the tubular member.

21. The drug delivery device of claim 20, wherein the axial displacement of the piston assembly is greater than the axial displacement of the tubular member by a factor of about 40.

22. The drug delivery device of claim 17, wherein the piston assembly is configured to convert axial displacement a first component into rotational displacement of a second component.

23. The drug delivery device of claim 17, wherein the axially fixed distal member comprises an annular void arranged to receive a distal end region of the tubular member therein.

24. The drug delivery device of claim 17, wherein the axially fixed distal member comprises a luer connection.

25. The drug delivery device of claim 17, wherein the axially fixed distal member forms an ejection chamber that is fluidly connected to the mixing chamber.

26. The drug delivery device of claim 17, wherein the piston assembly forms a central lumen that is in fluid communication with the mixing chamber.

27. A drug delivery system comprising:
a drug delivery device forming a mixing chamber therein;
a manifold releasably attachable to the delivery device, the manifold comprising a first vial configured to contain a first substance and a second vial configured to contain a second substance, the first and second vials being in fluid communication with one another such that when the first vial contains the first substance and the second vial contains the second substance the first and second substances can be combined to form a third substance; and
an fluid moving device in fluid communication with the manifold and arranged to combine the first and second substances when activated in a first mode and to deliver the third substance to the mixing chamber of the drug delivery device when activated in a second mode.

28. The drug delivery system of claim 27, further comprising a therapeutic agent disposed within the mixing chamber.

29. The drug delivery system of claim 28, wherein the therapeutic agent comprises a powdered substance.

30. The drug delivery system of claim 28, wherein the therapeutic agent component comprises an osteogenic agent.

31. The drug delivery system of claim 27, wherein at least one of the first and second substances comprises a liquid.

32. The drug delivery system of claim 27, wherein one of the first substance comprises water and the second substance comprises a concentrate.

33. The drug delivery system of claim 27, wherein the drug delivery device further comprises an agitator assembly disposed within the mixing chamber, the agitator assembly being configured to axially reciprocate within the mixing chamber.

34. The drug delivery system of claim 27, wherein the drug delivery device further comprises a piston assembly operably connected to a tubular member that defines the mixing chamber, the piston assembly being configured to axially displace the tubular member to reduce the volume of the mixing chamber.

35. The drug delivery system of claim 27, wherein the manifold comprises a cover to which the first and second vials are capable of being secured, the cover being slidably disposed within a cavity of the manifold.

36. The drug delivery system of claim 35, wherein the manifold comprises first and second needles extending from a surface opposite the cover, the first and second needles being capable of penetrating the first and second vials, respectively, when the cover is slid within the cavity toward the needles.

37. The drug delivery system of claim 27, wherein the fluid moving device comprises a syringe.

38. The drug delivery system of claim 27, wherein activating the fluid moving device in the first mode causes fluid to be withdrawn from one of the vials, and activating the fluid moving device in the second mode causes fluid to be introduced into one of the vials.

39. A method, comprising:
axially reciprocating an agitator assembly within a mixing chamber of a drug delivery device to mix a plurality of substances within the mixing chamber; and
after mixing the plurality of substances, ejecting the plurality of mixed substances from the mixing chamber.

40. The method of claim 39, wherein axially reciprocating the agitator assembly comprises rotating a rotary drive operably coupled to the agitator assembly.

41. The method of claim 40, wherein rotating the rotary drive comprises rotating the rotary drive a predetermined number of revolutions.

42. The method of claim 41, wherein the predetermined number of revolutions is equal to about 16 revolutions.

43. The method of claim 39, wherein ejecting the liquid composition comprises axially displacing a piston assembly extending within the drug delivery device.

44. The method of claim 43, wherein axially displacing the piston assembly causes axial displacement of a tubular member defining the mixing chamber relative to an axially fixed distal member of the drug delivery device.

45. The method of claim 44, wherein the axial displacement of the tubular member reduces a volume of the mixing chamber.

46. The method of claim 39, further comprising introducing at least one of the plurality of substances into the mixing chamber.

47. The method of claim 46, wherein introducing the at least one of the plurality of substances into the mixing chamber comprises actuating an air moving device that is fluidly connected to the mixing chamber.

48. The method of claim 39, further comprising combining first and second substances to form a third substance, and introducing the third substance into the mixing chamber.

49. A method for preparing bone cement, the method comprising:
providing a drug delivery system comprising a manifold and a delivery device configured for releasable attachment to the manifold;
reconstituting a bone morphogenetic protein powder to form a bone morphogenetic protein admixture in the manifold;
delivering the bone morphogenetic protein admixture from the manifold to the delivery device;
mixing the bone morphogenetic protein admixture with a calcium phosphate matrix contained within the delivery device to form a bone cement paste; and
ejecting the bone cement paste from the delivery device.

50. A drug delivery system comprising:
a manifold configured to contain a bone morphogenetic protein therein; and
a delivery device configured to contain a calcium phosphate matrix therein, and configured for releasable attachment to the manifold so that the bone morphogenetic protein can be delivered to the delivery device when the manifold contains the bone morphogenetic protein, the delivery device comprising
an agitator assembly configured to mix the calcium phosphate matrix and the bone morphogenetic protein when the delivery device contains the calcium phosphate matrix and the bone morphogenetic protein is delivered to the delivery device; and
an aperture defined in a distal end of the delivery device to allow contents to be ejected from the delivery device.
